(19) United States
(12) Patent Application Publication
(10) Pub. No.: US 2024/0065927 A1
(43) Pub. Date: Feb. 29, 2024

(54) ELECTRIC SPINE BACK RACK ORTHOPEDIC APPARATUS FOR ALLEVIATING BACK PAIN AND DECOMPRESSING FACET JOINTS AND SPINE VERTEBRAS

(71) Applicant: Bohdan Miroslaw Luklinski, Guildford (GB)

(72) Inventor: Bohdan Miroslaw Luklinski, Guildford (GB)

(21) Appl. No.: 17/895,497

(22) Filed: Aug. 25, 2022

(51) Int. Cl. A61H 15/00

(52) U.S. Cl. CPC A61H 15/0078; A61H 2015/005; A61H 2201/0149; A61H 2201/1207; A61H 2201/1623; A61H 2201/503; A61H 2205/081

(57) ABSTRACT

This present invention relates to an electric spine back pain relieving device. The device has a fixed frame that is configured to be integrated or retrofitted to backrest of a chair for providing support to spine of a user. The device has a frame and has an integrated electric source for providing mechanical pressure on facet joints or vertebras of spine of a human. The rigid frame carries a plurality of parallel rods attached to and crossing the space between the two longer arms of the frame. Each of the rod in the cervical, thoracic and lumbar regions carry a pair of barrel-shaped or spherical spindle nodules that are positioned below the transverse processes of the spine for alleviating pain. The electric source can be selectively activated using a push button and the device works on one of the two modes: single action mode and block action mode.

### FIELD OF THE INVENTION

The present invention relates generally to the field of orthopedic and back pain-relieving devices. More specifically, the present invention relates to a novel device that can be integrated or retrofitted in any chair for decompressing facet joints of spine of a human. A pair of spindle nodules abut the facet joints of spine for decompression and helps in alleviating back pain caused due to passive seating on a chair.

### BACKGROUND OF THE INVENTION

By way of background, human spine consists of a number of vertebrae, or units of bone, arranged in a vertical structure, also known as the spinal column. The spinal column consists of five distinct regions namely Cervical spine, Thoracic spine, Lumbar spine, Sacrum, and Coccyx. Further, the spine of a human performs two-fold functions—providing structural support for the body and allowing to move freely in six dimensions: flexion, extension, side flexion left/right and rotation. In order to achieve the support and motion, the vertebrae in the top three sections (Cervical spine, Thoracic spine, and Lumbar spine) must be allowed to move relative to one another, without compromising the structural rigidity of the back. This is achieved by the use of facet joints, which lock the vertebrae together whilst allowing for articulated movement. The joints are referred to as facet joints because they are formed when the faces of two bones come together.

Unfortunately, facet joints are prone to be compressed relatively easily. When the facet joints are compressed, the vertebrae in the spine can no longer move freely. Humans feel a stiff and experience back pain because of compressed facet joints. Various research has shown that up to 98% of all back pain is either caused by, or directly linked to compression of facet joints.

Backpain due to compression of facet joints is a common issue in seats like automobile, aviation, furniture and all sedentary positions. People who work in offices, drivers, pilots and people who commute on a daily basis are prone to back pain and discomfort due to compression of facet joints. Current seat structures do not address problems of back pain due compression of facet joints and therefore, people desire a method and a device for alleviating back pain by de-compressing the spine of a user.

Therefore, there exists a long felt need in the art for a device that solves the back pain problem by de-compressing the spine while passive seating occurs. There is also a long felt need in the art for a back pain alleviating device that can be integrated in seats in automobiles, aviation and furniture. Additionally, there is a long felt need in the art for a back support device that improves health of spine. Moreover, there is a long felt need in the art for a device that conforms to curvature of spine of human. Finally, there is a long felt need in the art for a device that performs de-compression of facet joints.

The electric spine back rack device of the present invention accomplishes all of the forgoing objectives and provides users with a spinal electric device for relieving back pain. The spindles of the device are activated mechanically causing the motion of the whole spine/each joint causing decompression of facet joints. The spindle penetration of the device depends on an individual person tissue thickness/mass layers tissues and muscles and the device is effective to perform, irrespective of spinal joint distribution upon spindle.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an electric spine orthopedic device made of plastic or similar material and is configured to integrate or retrofit to a chair for providing relief from back pain to people sitting in the chair. The device further comprising a rectangular frame having a pair of arms, a plurality of rods attached to and crossing the space between said pair of arms, the plurality of rods forming groups located in the position of cervical, thoracic, lumbar, and pelvic regions of spine of a human, each rod in said cervical, thoracic, and lumbar groups having a pair of spindle nodules, wherein the nodules of each pair are positioned 7.5 cm apart and are round or barrel shaped, an electric source for providing electric power to create mechanical pressure in said nodules, and said mechanical pressure is applied in one of said cervical, thoracic, and lumbar regions for a duration of up to six seconds or on a plurality of facet joints simultaneously.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the electric spine back rack device of the present invention in accordance with the disclosed architecture;

FIG. 2 illustrates a block diagram illustrating different modes of the electric spine back rack orthopedic device of the present invention in accordance with the disclosed architecture;

FIG. 3 illustrates a close view of a nodules of a spindle used in the orthopedic spine back rack device of the present invention in accordance with the disclosed architecture;

FIG. 4 illustrates a perspective view of one potential embodiment of the electric orthopedic back rack device 100 of the present invention being installed in a chair in accordance with the disclosed architecture;

FIG. 5 illustrates a perspective view of a user sitting on the chair 400 with installed device 100 in accordance with the disclosed architecture; and

FIG. 6 illustrates a close view of position of spindle nodules positioned and abutting the facet joints of spine in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. The well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a device that solves the back pain problem by de-compressing the spine while passive seating occurs. There is also a long felt need in the art for a back pain alleviating device that can be integrated in seats in automobiles, aviation and furniture.

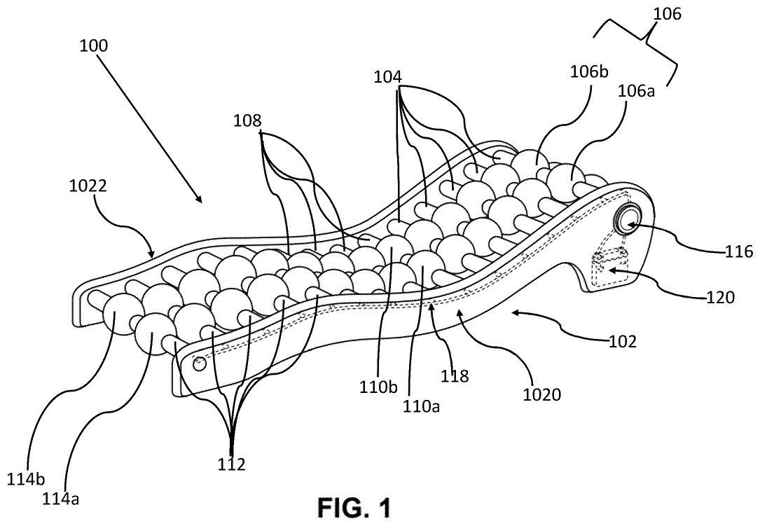

Referring initially to the drawings, FIG. 1 illustrates a perspective view of one potential embodiment of the electric back rack device of the present invention in accordance with the disclosed architecture. The electric spine back rack device 100 of the present invention is an orthopedic spine back rack device for decompressing facet joints and helping in alleviating back pain. The device 100 is designed to support the spine of a human and conforms to shape of the spine. More specifically, the device 100 provides decompression of facet joint on cervical, thoracic and lumbar regions of the spine without any side effects. The electric spine back rack orthopedic device 100 provides complete relief from skeletal pain and tension and helps in increasing flexibility, mobility, and range of motion.

The device 100 mirrors natural shape of the human spine and has a frame 102 made of a durable and hard material like heavy-duty plastic. The frame 102 has a first group of rods 104 wherein each rod of the first group of rods 104 carry a spindle 106 including a pair of barrel shaped nodules 106a, 106b for applying pressure on facet joints. For clarity, nodules of only one of the rods are annotated. More specifically, the spindle nodules 106a,106b are used for decompressing facet joints positioned between on the cervical portion of the spinal column.

Similarly, a second group of rods 108 is positioned below the first group of rods 104 wherein each rod of the second group of rods 108 carry a spindle 110 including a pair of barrel shaped nodules 110a, 110b for compressing facet joints of thoracic column of the spine. A third group of rods 112 having a spindle 114 including a pair of barrel shaped nodules 114a, 114b in each rod is used for decompressing lumbar column of the spine of a human. The pair of spindles in each rod are positioned such that the spindles abut the facet joints of the spine.

The first group of rods 104 and the third group of rods 112 are positioned on convex shape of the frame 102 and the second group of rods 108 are positioned on concave shape of the frame 102. Further, each of the rod of the frame 102 extends between a first arm 1020 and a second arm 1022.

The orthopedic device 100 has a push button 116 positioned on one of the arms of the frame 102 and is coupled to a circuit 118 passing along the frame 102 wherein the button 116 is configured to pass electric current from an integrated battery 120 to one or more pair of spindles 106, 110, 114 for applying spindle penetration for decompressing facet joints. In the preferred embodiment, the depth of the spindle penetration applied by the mechanical pressure can be up to 10 mm transversely depending on an individual person tissue thickness/mass layers tissues and muscles.

The nodules in each spindle have a transverse distance of 7.5 cm therebetween and each pair of the nodules have the same distance allowing a symmetric effect on the spine of a user. The orthopedic device 100 is configured to put mechanical pressure for decompressing facet joints in one direction i.e., either from top of the spine to bottom of the spine (exerting mechanical pressure from first group of rods 104 first to the third group of rods 112 at the end) or from the bottom of the spine to the top of the spine (exerting mechanical pressure from the third group of rods 112 at the end to the first group of rods 104). When the spindles penetrate in the spine, each vertebra is decompressed individually or several vertebras are decompressed simultaneously as described later in the disclosure.

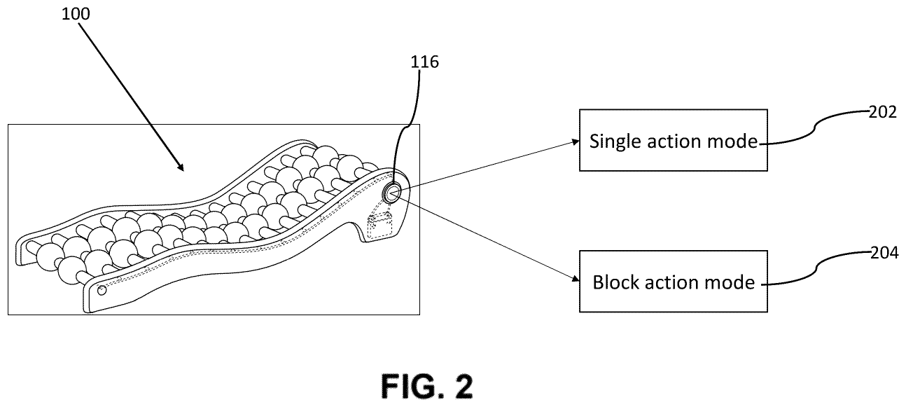

FIG. 2 illustrates a block diagram illustrating different modes of the electric orthopedic device of the present invention in accordance with the disclosed architecture. The device 100 is configured to work in two modes: a single action mode 202 and a block action mode 204. The push button 116 may be used for selecting one of the two modes 202,204 for decompressing facet joints of the spine. In single action mode 202, a pressure is applied to any section of the spine i.e., one of cervical, thoracic or lumbar section of the spine at one time. As an example, in single action mode 202, the pressure is applied to facet joints of L1 to L5 vertebras of lumbar column of the spine. The pressure is configured to be applied for up to six seconds and can be applied in a constant or intermittent manner.

In block action mode 204, a set of five spindles are activated simultaneously allowing a plurality of vertebras being pressurized allowing decompressing of different facet joint positioned in different sections of the spine. In the block action mode 204, different sets of four spindles are consecutively pressurized allowing decompression of facet joints.

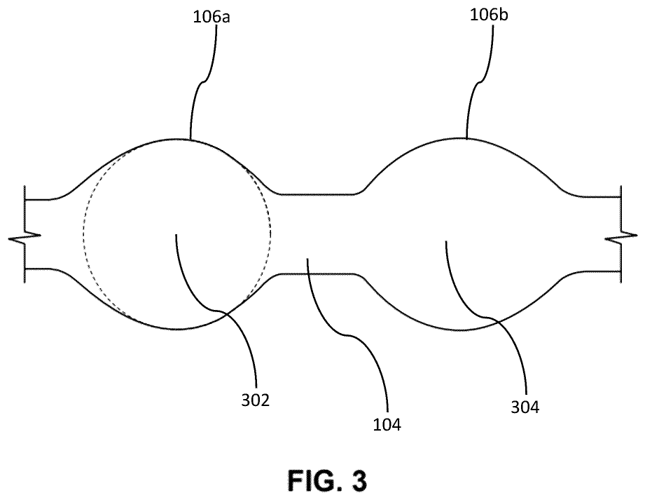

FIG. 3 illustrates a close view of a nodules of a spindle used in the orthopedic spine back rack device of the present invention in accordance with the disclosed architecture. The nodules 106a,106b have a diameter of about 48 mm and are round or barrel shaped such that when a user sits against the device 100, the nodules abut against the facet joints or vertebras of the spine. The width of the rod 104 is about 14 mm and is uniform along the length.

The distance between the centers 302,304 of the nodules 106a,106b is about 75 mm which is designed specifically to match the gap/distance between the human spine facet joints that runs through the spine of a human. It should be noted that in the present embodiment, the nodules 106a,106b are exemplary but all the nodules used in the device 100 may have same shape and sizes allowing a uniform mechanical pressure being applied on the facet joints/vertebras of the spine of a user.

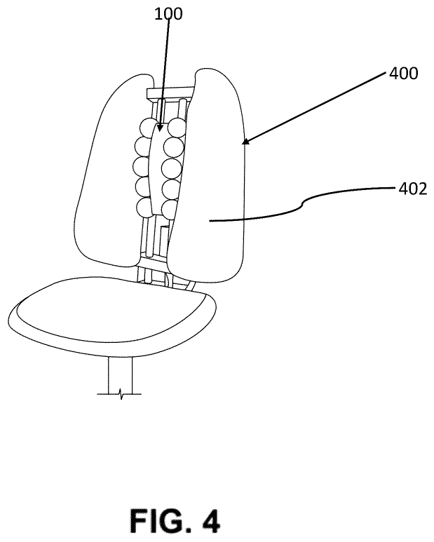

FIG. 4 illustrates a perspective view of one potential embodiment of the electric orthopedic spine back rack device 100 of the present invention being installed in a chair in accordance with the disclosed architecture. The spine back rack device 100 is configured to be installed in automobiles, airplanes, office seats and more for allowing decompression of facet joints/vertebras of spine of a user sitting on a seat with installed device 100.

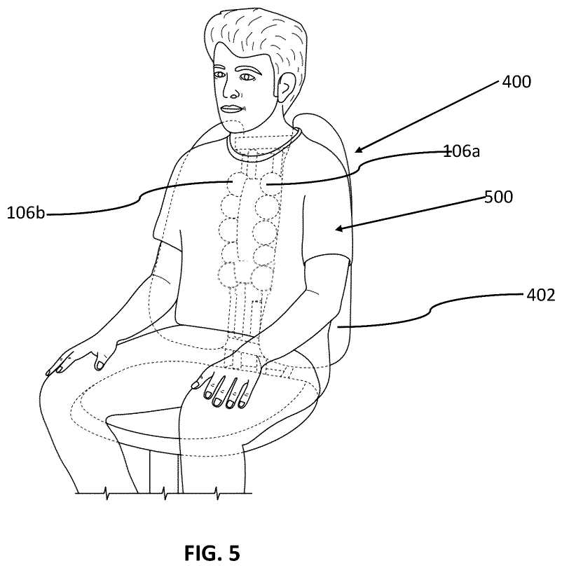

As illustrated in the exemplary chair 400, the device 100 is positioned in the backrest 402 orientation such that the nodules of the spindles abut against the facet joint of a user sitting on the chair 400 as illustrated in FIG. 5. In one embodiment, the device 100 is integrated into the chair 400 and alternatively, the device 100 can be retrofitted to the chair 400.

It should be appreciated that based on the design of the chair 400 and requirements of a user, the number of spindles and the rods in the frame can vary and even the mechanical pressure applied by the nodules can vary. The device 100 can work in both single action mode and block action mode described in FIG. 2.

FIG. 5 illustrates a perspective view of a user sitting on the chair 400 with installed device 100 in accordance with the disclosed architecture. A user 500 can sit on the chair 400 having integrated device 100 without any discomfort while having support on the spine where the nodules abut the facet joints of the spine. As illustrated, the exemplary nodules 106a,106b are installed such on the backrest 402 of the chair 400 such that the nodules exert mechanical pressure for decompressing the facet joints/vertebras and helping in alleviating the back pain.

Other shapes, designs and configurations of the electric orthopedic spine back rack 100 are contemplated and fall within the scope of the invention without affecting the utility of the device. Since the device can be installed in any type of seat, therefore, can be used in automobiles, offices, airplanes and more. The device 100 come in different sizes, shapes, materials and more.

The spine back rack car seat support works on back during travelling or resting. The device can be detachably attached and can be used for all forms of transport, from cars and trucks to trains and airplanes.

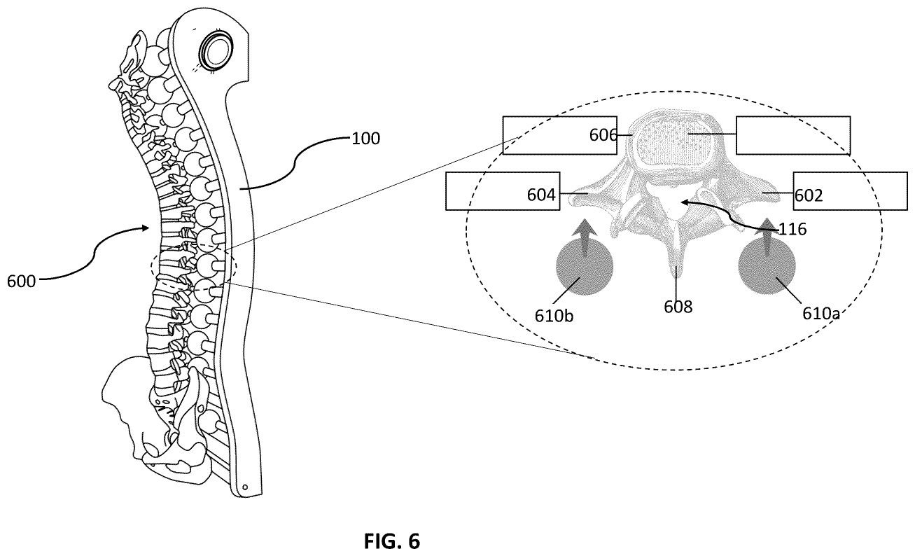

FIG. 6 illustrates a close view of position of spindle nodules positioned and abutting the facet joints of spine in accordance with the disclosed architecture. The exemplary nodules 610a,610b of a spindle are configured to push up on one or both sides 602,604 of the spine 600. The wooden nodules 610a,610b push up under the transverse processes 602,604. In turn, this lifts the individual vertebrae 606, causing the vertebra 606 to pivot about the facet joints 608. As a result of this, the joints 608 is decompressed, and the spine lengthens. Accordingly, the joint mobility and soft-tissue elasticity increases and stiffness is reduced.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “electric back rack device”, “back rack device”, “electric orthopedic device”, “orthopedic device” and “device” are interchangeable and refer to the electric spine back rack device 100 of the present invention.

Notwithstanding the forgoing, the electric spine back rack device 100 of the present invention can be of any suitable size and configuration as is known in the art without affecting the overall concept of the invention, provided that it accomplishes the above stated objectives. One of ordinary skill in the art will appreciate the electric spine back rack device 100 as shown in the FIGS. are for illustrative purposes only, within the scope of the present disclosure. Although the dimensions of the electric back rack device 100 important design parameters for user convenience, the electric spine back rack device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An electric spine back rack orthopedic device comprising:
a rectangular frame having a pair of arms;
a plurality of rods attached to and crossing the space between said pair of arms;
the plurality of rods forming at least three groups located in the position of cervical, thoracic, and lumbar regions of spine of a human;
each rod in said groups of cervical, thoracic, and lumbar regions having a pair of spindle nodules, wherein the nodules of each pair are positioned 7.5 cm apart;
an electric source for providing electric power to create mechanical pressure in said nodules; and
said mechanical pressure is applied in one of said cervical, thoracic, and lumbar regions for a duration of up to six seconds.

2. A frame for support of spine of a human comprising:
a rectangular frame having a pair of arms;
a plurality of rods attached to and crossing the space between said pair of arms;
the plurality of rods forming at least three groups located in the position of cervical, thoracic, and lumbar regions of spine of a human;
each rod in said groups of cervical, thoracic, and lumbar regions having a pair of spindle nodules, wherein the nodules of each pair are positioned 7.5 cm apart;
an electric source for providing electric power to create mechanical pressure in said nodules; and
said mechanical pressure is applied in on five pairs of said lumbar section spindle nodules enabling several vertebras undergoing pressure simultaneously.

3. The frame of claim 2, wherein the spindle nodules are round or barrel shaped.

4. The frame of claim 2, wherein the electric source is an integrated battery.

5. The frame of claim 2, wherein the electric power is transferred on pushing a push button disposed on one of said arms.

6. The frame of claim 2, wherein the electric power is transferred from the battery through a wired circuit.

7. The frame of claim 2, wherein the frame is integrated in a chair at the time of manufacturing of the chair.

8. The frame of claim 2, wherein the frame is retrofitted to backrest of a chair.
